(19) United States
(12) Patent Application Publication
(10) Pub. No.: US 2026/0191994 A1
(43) Pub. Date: Jul. 9, 2026

(54) Vivo gene editing of Tau locus via liponanoparticle delivery

(71) Applicant: The Regents of the University of California, Oakland, CA (US)

(72) Inventors: Niren Murthy, Berkeley, CA (US); Andrea Gomez, Berkeley, CA (US); Jennifer Doudna, Berkeley, CA (US); Rohit Sharma, Berkeley, CA (US); Yung Yi Hsiao, Berkeley, CA (US); Fyodor Urnov, Berkeley, CA (US); Elizabeth Stahl, Berkeley, CA (US); Viviana Salinas-Rios, Berkeley, CA (US)

(73) Assignee: The Regents of the University of California, Oakland, CA (US)

(21) Appl. No.: 19/566,978

(22) Filed: Mar. 14, 2026

Related U.S. Application Data
(63) Continuation of application No. PCT/US24/47351, filed on Sep. 19, 2024.
(60) Provisional application No. 63/583,857, filed on Sep. 19, 2023.

(51) Int. Cl. A61K 48/00; A61K 9/00; A61K 9/1272; A61K 9/51; A61K 38/17; A61P 25/28; C12N 9/22; C12N 15/11; C12N 15/88

(52) U.S. Cl. CPC A61K 48/005; A61K 9/0085; A61K 9/1272; A61K 9/5123; A61K 38/1709; A61K 48/0075; A61P 25/28; C12N 9/226; C12N 15/111; C12N 15/88; C12N 2310/20

(57) ABSTRACT

Lipid nanoparticles are used to deliver various types of nuclei acids to different tissues in vivo, providing a novel tool for gene editing.

### CROSS-REFERENCES TO RELATED APPLICATIONS

This application is a continuation of PCT/US24/47351, filed Sep. 19, 2024, which claims priority to U.S. Provisional Application No. 63/583,857; filed Sep. 19, 2023, the disclosures of which are hereby incorporated by reference in its entirety for all purposes.

### REFERENCE TO A SEQUENCE LISTING

A Sequence Listing in XML format is incorporated by reference into the specification. The name of the XML file containing the Sequence Listing is B24-026-2US.xml. The XML file is 8,299 bytes and was created on Mar. 14, 2026.

### INTRODUCTION

We developed a new class of lipid nanoparticle (LNP) effective in delivering various types of nucleic acids to different tissues in vivo, providing a novel tool for gene editing.

### SUMMARY OF THE INVENTION

Disclosed is a new class of lipid nanoparticle (LNP) effective in delivering various types of nucleic acids to different tissues in vivo, providing a novel tool for gene editing.

The invention provides methods and compositions for in vivo gene editing of Tau locus via liponanoparticle delivery.

In aspects and embodiments the invention provides:
1. A lipid nanoparticle (LNP) configured for delivering a nucleic acid to the brain, the LNP comprising an ionizable (acid-degradable) lipid.
2. The LNP of claim 1, further comprising gene editing molecules, like CRISPR Cas9 and a gRNA.
3. The LNP of claim 1, further comprising gene editing molecules, like CRISPR Cas9 and a gRNA, configured to target Tau.
4. The LNP of claim 1, further comprising epigenome editing molecules, such as epigenome modifying polypeptides, such as a catalytically inactive Cas9 fused to an epigenome modifying domain.
5. The LNP of claim 1, further comprising epigenome editing molecules, such as epigenome modifying polypeptides, such as a catalytically inactive Cas9 fused to an epigenome modifying domain, configured to target Tau.
6. The LNP of claim 1, further comprising a helper lipid, such as DOPE, cholesterol, and a DMG PEG (dimyristoyl glycerol polyethyleneglycol).
7. The LNP of claim 5, further comprising a helper lipid, such as DOPE, cholesterol, and a DMG PEG (dimyristoyl glycerol polyethyleneglycol).
8. The LNP of claim 1, wherein the ionizable lipid is selected from: Dlin, Lipid 9, L-319, L-A6, cKK-E12, Lipid C24, ALC-0315, OF-02, Lipid A9, Lipid CL1, YK-009, ALC-0315 analogous-1, AA-T3A-C12, CL4F8-6, Lipid III-45.
9. The LNP of claim 5, wherein the ionizable lipid is selected from: Dlin, Lipid 9, L-319, L-A6, cKK-E12, Lipid C24, ALC-0315, OF-02, Lipid A9, Lipid CL1, YK-009, ALC-0315 analogous-1, AA-T3A-C12, CL4F8-6, Lipid III-45.
10. The LNP of claim 1, further comprising an azide-benzaldehyde acetal cholesterol.
11. The LNP of claim 5, further comprising an azide-benzaldehyde acetal cholesterol.
12. The LNP of claim 1, further comprising a cerebroside (a lipid comprising a cerebroside head group).
13. The LNP of claim 5, further comprising a cerebroside.
14. The LNP of claim 1, further comprising a cerebroside selected from: monoglycosyl and oligoglycosylceramides having a mono or polysaccharide bonded glycosidically to the terminal OH group of ceramide, particularly with sphingosine as the main long-chain base present in the ceramide.
15. The LNP of claim 1, wherein lipid formulation is selected from: F5, L-4, L-5, L-6, L-10, L-14, L-18, L-24, L-25, L-36, L-40, L-42, L-45, L-46, L-47
16. In vivo gene editing of a Tau locus via liponanoparticle delivery using a LNP of any of claims 1-15.
17. Use of an LNP of any of claims 1-15, to deliver editing components to the central nervous system.
18. Use of an LNP of any of claims 1-15, to prevent and/or treat brain diseases like Alzheimer's disease or Frontotemporal Dementia.
19. Use of an LNP of any of claims 1-15, to deliver gene editing molecules to neurons in vivo.
20. Use of an LNP described of any of claims 1-15, to deliver gene editing molecules to primary neurons in vitro.
21. Use of an LNP of any of claims 1-15, containing mRNA and gRNA that can edit disease causing genes in the brain after an intracranial injection.
22. Use of an LNP of any of claims 1-15, containing mRNA and gRNA that can edit a Tau locus via LNP mediated mRNA delivery.
23. Use of an LNP of any of claims 1-15, containing mRNA and gRNA that can edit a Tau locus with a modified gRNA encapsulated in LNPs that comprise Cas9 mRNA.
24. Use of an LNP of any of claims 1-15, to deliver Cas9 to neurons.
25. Use of an LNP of any of claims 1-15, for delivery to neurons of Cas9 mRNA+Tau5 gRNA.

In additional aspects and embodiments the invention provides:
1. In vivo gene editing of a Tau locus via liponanoparticle delivery.
2. A lipid nanoparticle (LNP) comprising a lipid comprising a cerebroside head group.
3. An LNP herein comprising editing components, like CRISPR Cas9 and a gRNA.
4. An LNP herein comprising editing components configured for epigenome editing, such as use of a catalytically inactive Cas9 fused to an epigenome modifying domain.
5. An LNP herein comprising an azide-benzaldehyde acetal cholesterol.
6 An LNP herein comprising a cerebroside, including monoglycosyl and oligoglycosylceramides having a mono or polysaccharide bonded glycosidically to the terminal OH group of ceramide, particularly with sphingosine as the main long-chain base present in the ceramide.
7. A lipid formulation, such as the example referred to as F5 herein, comprising a cerebroside in a bilayer forming lipid with or without acid degradable polyethylene glycol (PEG), attached, which may be packaged together with gene editing components, like CRISPR Cas9 and a gRNA targeting an endogenous locus, such as Tau.
8. Use of an LNP described herein to deliver editing components to the central nervous system.
9. Use of an LNP herein to prevent and/or treat brain diseases like Alzheimer's Disease or Frontotemporal Dementia.
10. Use of an LNP herein to deliver gene editing molecules to neurons in vivo.
11. Use of an LNP described herein to deliver gene editing molecules to primary neurons in vitro.
12. Use of an LNP herein containing mRNA and gRNA that can edit disease associated genes in the brain after an intracranial injection.
13. Use of an LNP described herein containing mRNA and gRNA that can edit a Tau locus via LNP mediated mRNA delivery.
14. Use of an LNP described herein containing mRNA and gRNA that can edit a Tau locus with a modified gRNA encapsulated in the LNPs that comprise Cas9 mRNA.
15. Use of an LNP herein to deliver Cas9 to neurons.
16. Use of an LNP-PEG described herein for delivery to neurons of Cas9 mRNA+Tau5 gRNA
17. Methods, processes, compositions and systems disclosed herein, including those of the figures.

In additional aspects and embodiments the invention provides:

### LNP Details

1. A lipid nanoparticle (LNP) comprising a lipid comprising a cerebroside head group.
2. The LNP of claim 1, further comprising a cerebroside, including monoglycosyl and oligoglycosylceramides having a mono or polysaccharide bonded glycosidically to the terminal OH group of ceramide.
3. The LNP of claim 1, further comprising a cerebroside, including monoglycosyl and oligoglycosylceramides having a mono or polysaccharide bonded glycosidically to the terminal OH group of ceramide, with sphingosine as the main long-chain base present in the ceramide.
4. The LNP of claim 1, further comprising an azide-benzaldehyde acetal cholesterol.
5. The LNP of claim 1, configured for central nervous system or brain delivery.

### Delivery of Editors or Epieditors Using the LNPs

6. The LNP of claim 1, further comprising RNA guided effector polypeptide components for nucleic acid cleavage or epigenome modifying polypeptides configured for delivering epigenome modifying polypeptides.
7. The LNP of claim 6, where said epigenome modifying polypeptides consist of catalytically inactive CRISPR Cas9 fused to an epigenome modifying domains.
8. The LNP of claim 6, where said RNA guided effector polypeptide comprises a CRISPR Cas9 and a gRNA.

### Formulation/Composition

7. A lipid formulation, such as the example referred to as F5 herein, comprising a cerebroside in a bilayer forming lipid with or without acid degradable polyethylene glycol (PEG) attached, which may be packaged together with gene editing components, like CRISPR Cas9 and a gRNA.

### General use of LNP

8. Use of the LNP described in any one of claims 1-6 to deliver RNA guided effector polypeptide components for nucleic acid cleavage or epigenome modifying polypeptides components to the central nervous system.

9. Use of the lipid formulation of claim 7 to deliver RNA guided effector polypeptide components for nucleic acid cleavage or epigenome modifying polypeptides components to the central nervous system.

10. Use of the LNP or lipid formulation of claim 8 or 9 to treat brain diseases like Alzheimer's Disease or Frontotemporal Dementia.

11. Use of the LNP or lipid formulation of claim 8 or 9 to deliver gene editing molecules to neurons in vitro or in vivo.

12. Use of the LNP or lipid formulation of claim 8 or 9 to edit disease causing genes in the brain after an intracranial injection.

### Use Specific to Tau

13. Use of the LNP or lipid formulation of claim 8 or 9 to perform in vivo gene editing of a Tau locus.

14. Use of the LNP or lipid formulation of claim 8 or 9 to deliver RNA guided effector polypeptide components for nucleic acid cleavage or epigenome modifying polypeptides components to the brain that target the Tau locus.

15. Use of an LNP or lipid formulation of claim 8 or 9 that comprises mRNA and gRNA that can edit a Tau locus with a modified gRNA encapsulated in LNPs that comprise Cas9 mRNA.

16. Use of an LNP formulation of claim 7 for delivery to neurons of mRNA encoding Cas9 and a Tau5 specific gRNA.

The invention encompasses all combinations of the aspects and particular embodiments recited herein, as if each combination had been laboriously recited.

### BRIEF DESCRIPTION OF THE DRAWINGS

FIGS. 1A-C. LNP delivery of Cas9 mRNA and gRNA targeting the Tau locus in primary mouse neurons. DNA was extracted 3 days post transfection and sequenced with NGS. Percent (%) editing of the Tau locus was quantified using CRISPRESSO2 comparing LNP delivery vs AAV delivery of gRNA. DIV3 transfection, 1.5 ug Cas9 mRNA+cutting gRNA. DNA extraction 3 days post-transfection (7 days for AAV) (FIG. 1A). Percent indel formation following editing using different LNP compositions in comparison with AAV delivery (FIG. 1B) shows that the F5 formulation resulted in higher editing frequency. Next Generation Sequencing (NGS) results demonstrating indels following editing (FIG. 1C); the displayed reference sequence is: TGACGAGAAGAAAGCCAAGGTAAGCTACTGACCCGCCGGT (SEQ ID NO:1)

FIGS. 2A-B. LNP delivery of Cas9 in Wildtype Primary Mouse Neurons: detection of INDELs using LNPs targeting Tau5. FIG. 2A panels depict morphology of the cells following treatment with either the F5 LNPs or F5-PEG LNPs. FIG. 2B depicts comparison of F5 LNP with or without PEG, in terms of toxicity to neurons and editing in vitro. 560 ng Cas9 mRNA+560 ng Tau5 gRNA.

FIGS. 3A-C. LNP delivery of Cas9 mRNA and gRNA targeting the Tau locus using F5-PEG in vivo. 3 μL of LNPs was stereotaxically injected into the CA1 of mouse hippocampus using the coordinates AP: −2, ML: +/−1.3, DV: −1.3. DNA was extracted 7 days post injection and sequenced with NGS. Percent editing of the Tau locus was quantified using CRISPRESSO2. 3 uL, F5+PEG: 560 ng Cas9 mRNA+560 ng Tau5 gRNA. 3 animals, DNA extraction 7 days post-surgery. FIG. 3A shows the site of delivery into the brain. FIG. 3B depicts editing observed following ipsilateral injection of the editing reagents versus editing in the contralateral side following buffer injection. FIG. 3C shows the occurrence of indels following delivery; the aligned sequences are:

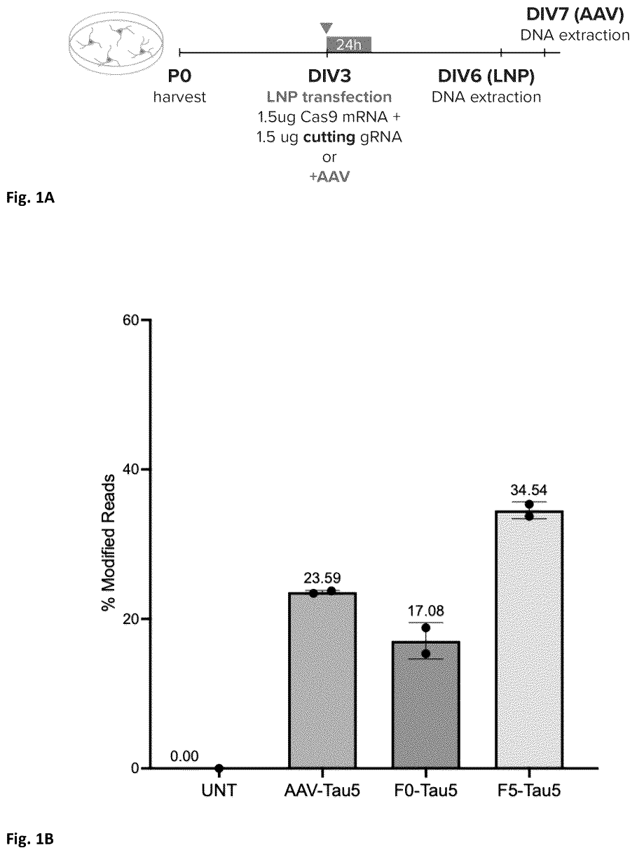

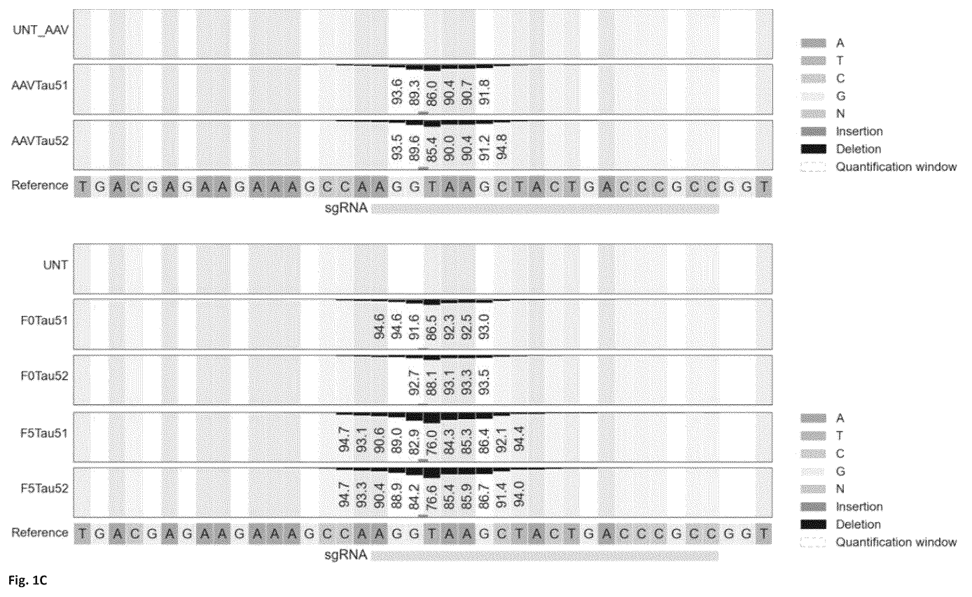

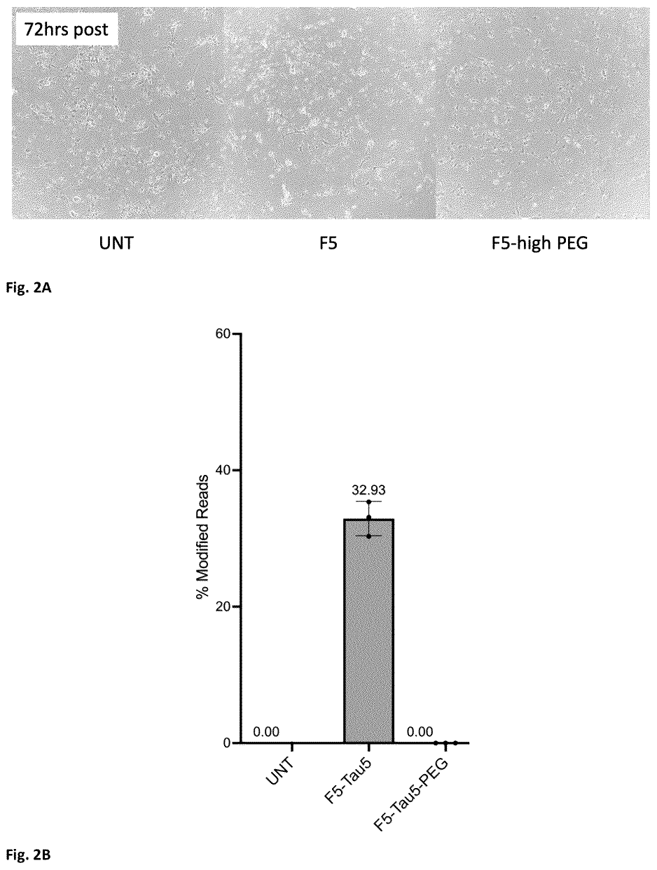

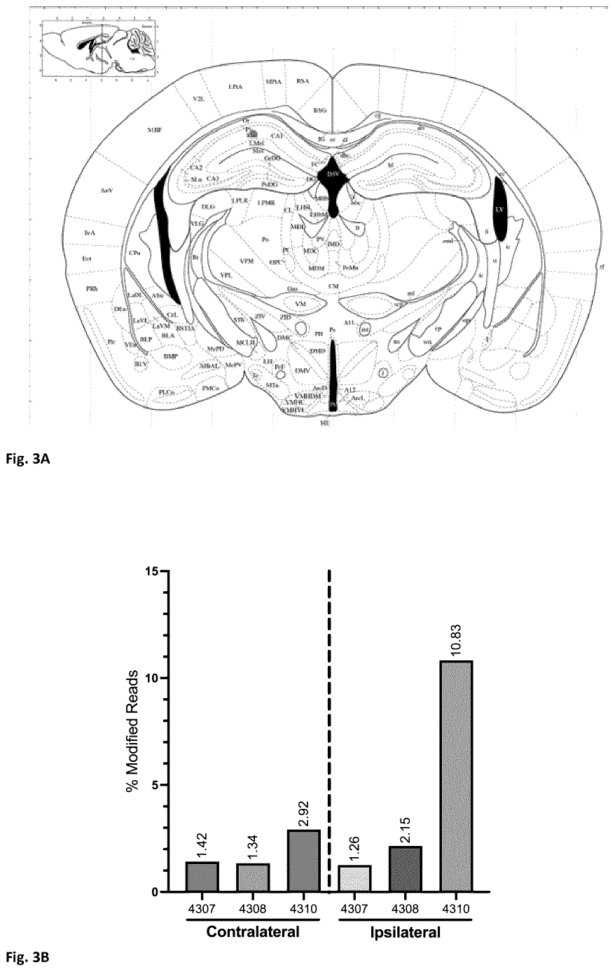

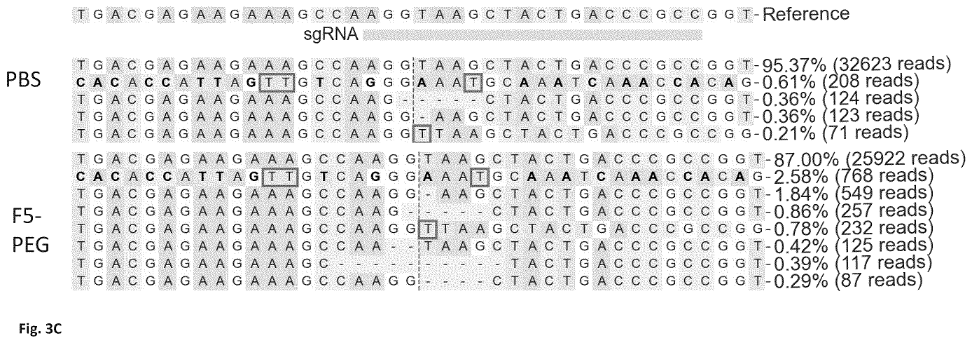

(SEQ ID NO: 1)
TGACGAGAAGAAAGCCAAGGTAAGCTACTGACCCGCCGGT
(SEQ ID NO: 1)
TGACGAGAAGAAAGCCAAGGTAAGCTACTGACCCGCCGGT
(SEQ ID NO: 2)
CACACCATTAGTTGTCAGGGAAATGCAAATCAAACCACAG
(SEQ ID NO: 3)
TGACGAGAAGAAAGCCAAG-----CTACTGACCCGCCGGT
(SEQ ID NO: 4)
TGACGAGAAGAAAGCCAAGG-AAGCTACTGACCCGCCGGT
(SEQ ID NO: 5)
TGACGAGAAGAAAGCCAAGGTTAAGCTACTGACCCGCCGG
(SEQ ID NO: 1)
TGACGAGAAGAAAGCCAAGGTAAGCTACTGACCCGCCGGT
(SEQ ID NO: 2)
CACACCATTAGTIGTCAGGGAAATGCAAATCAAACCACAG
(SEQ ID NO: 4)
TGACGAGAAGAAAGCCAAGG-AAGCTACTGACCCGCCGGT
(SEQ ID NO: 3)
TGACGAGAAGAAAGCCAAG-----CTACTGACCCGCCGGT
(SEQ ID NO: 5)
TGACGAGAAGAAAGCCAAGGTTAAGCTACTGACCCGCCGG
(SEQ ID NO: 6)
TGACGAGAAGAAAGCCAA--TAAGCTACTGACCCGCCGGT
(SEQ ID NO: 7)
TGACGAGAAGAAAGC--TACTGACCCGCCGGT
(SEQ ID NO: 8)
TGACGAGAAGAAAGCCAAGG----CTACTGACCCGCCGGT.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

For additional description of embodiments of the invention, see drawings and accompanying descriptions. Alternative, suitable cationic, ionizable lipids and LNP components are known in the art, e.g. Sun et al., Pharm Res. 2023; 40 (1): 27-46. Structure and Function of Cationic and Ionizable Lipids for Nucleic Acid Delivery; Khare et al., Adv Drug Deliv Rev. 2023 June: 197:114861, Lipid nanoparticle-mediated drug delivery to the brain. An exemplary

Cerebrosides are neutral compounds comprising a ceramide (sphingosine fatty acid) and a monosaccharide bound by a β-glycosidic bond, e.g.:

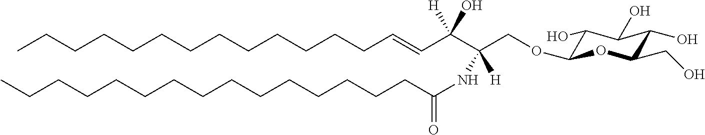

Azide-benzaldehyde acetal cholesterols are described in WO2024/049400, incorporated herein by reference, e.g.:

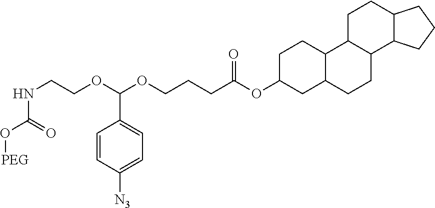

We developed a new class of lipid nanoparticle (LNP) effective in delivering various types of nucleic acids to different tissues in vivo, as demonstrated in mouse models, providing a novel tool for gene editing. The lipid formulation, such as the example referred to as F5 herein, contained a cerebroside in a bilayer forming lipid with or without acid degradable polyethylene glycol (PEG) attached, which may be packaged together with gene editing components, like CRISPR Cas9 and a gRNA targeting an endogenous locus, such as Tau. Thus the invention provides a therapeutically relevant intervention in the development of pathological states associated with Tau aggregate formation, wherein Tau dysregulation is a pathological feature of Alzheimer's Disease and Frontotemporal Dementia.

We demonstrate that editing of the Tau locus is possible with a modified gRNA encapsulated in LNPs that contain Cas9 mRNA. The Tau locus has never been edited before in vivo via LNP mediated mRNA delivery. The type of LNP used is innovative, as is the gRNA and genomic target.

Gene modulation can similarly be achieved using epigenome modifying polypeptides, see Thakore et a., Nat Methods, 2016 February; 13 (2): 127-37. In embodiments a cell, such as a neuron, is contacted with a lipid nanoparticle comprising an mRNA encoding a catalytically inactive RNA-guided endonuclease such as SpCas9 fused to an epigenome modifying polypeptide domain (including, but not limited to, KRAB, DNMT) and a guide RNA that directs the resulting protein fusion to an endogenous gene wherein expression of said gene is lowered. In further embodiments the gene is microtubule-associated protein tau (MAPT) encoding Tau and the neuron is in an individual with (e.g., treatment), or at risk of (e.g., prevention), a neurodegenerative disease such as Alzheimer's Disease or Frontotemporal Dementia.

The invention deploys a lipid containing a cerebroside head group in the LNPs, and these LNPs readily transfect neurons. This lipid has never used before for LNP formulation.

In embodiments the LNPs disclose herein are used prevent or treat brain diseases like Alzheimer's Disease or Frontotemporal Dementia, wherein an LNP containing mRNA and gRNA that can edit disease-associated genes in the brain after an intracranial injection.

As used herein, “gene editing components” refers to compounds capable of targeted editing of nucleic acids that encode gene sequences. Gene editing components can refer to CRISPR/Cas systems, Zinc Finger Nuclease (ZFN), Transcription activator-like effector nucleases (TALENs), Meganucleases, Mega-TALS and the like. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas 13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas 14b polypeptide, or a Cas14c polypeptide. Also suitable for use is a variant CRISPR-Cas effector polypeptide, where the variant CRISPR-Cas effector polypeptide has reduced nucleic acid cleavage activity. Also suitable for use is a CRISPR-Cas effector fusion polypeptide comprising: i) a CRISPR-Cas effector polypeptide is a variant that has reduced nucleic acid cleavage activity; and ii) a heterologous fusion polypeptide. In some cases, the heterologous fusion polypeptide is a protein modifying enzyme. In some cases, the heterologous fusion polypeptide is a nucleic acid modifying enzyme. In some cases, the heterologous fusion polypeptide is a reverse transcriptase. In some cases, the heterologous fusion polypeptide is a cytidine deaminase. In some cases, the heterologous fusion polypeptide is an adenine deaminase. In some cases, the heterologous fusion polypeptide is a transcription factor. In some cases, the heterologous fusion polypeptide is a transcription activator. In some cases, the heterologous fusion polypeptide is a transcription repressor. Suitable protein-modifying enzymes and nucleic acid modifying enzymes are described in detail below. For example, in some cases, the nucleic acid modifying enzyme is a cytidine deaminase. In some cases, the nucleic acid modifying enzyme is an adenosine deaminase. In some cases, the nucleic acid modifying enzyme is a prime editor. In some cases, the CRISPR-Cas effector polypeptide comprises one or more nuclear localization signals. Examples of CRISPR-Cas effector polypeptides are CRISPR-Cas endonucleases (e.g., class 2 CRISPR-Cas effector polypeptide such as a type II, type V, or type VI CRISPR-Cas effector polypeptide). Where a CRISPR-Cas effector polypeptide has endonuclease activity, the CRISPR-Cas effector polypeptide may also be referred to as a “CRISPR-Cas endonuclease.” A CRISPR-Cas effector polypeptide can also have reduced or undetectable endonuclease activity. A CRISPR-Cas effector polypeptide can also be a fusion CRISPR-Cas effector polypeptide comprising a heterologous fusion partner. In some cases, a suitable CRISPR-Cas effector polypeptide is a class 2 CRISPR-Cas effector polypeptide. In some cases, a suitable CRISPR-Cas effector polypeptide is a class 2 type II CRISPR-Cas effector polypeptide (e.g., a Cas9 protein). In some cases, a suitable CRISPR-Cas effector polypeptide is a class 2 type V CRISPR-Cas endonuclease (e.g., a Cpf1 protein, a C2c1 protein, or a C2c3 protein). In some cases, a suitable CRISPR-Cas effector polypeptide is a class 2 type VI CRISPR-Cas effector polypeptide (e.g., a C2c2 protein; also referred to as a “Cas13a” protein). Also suitable for use is a CasX protein. Also suitable for use is a CasY protein.

As used herein, “Epigenome modifying polypeptides” refers to fusion proteins configured to target a target nucleic acid or a protein produced therefrom at the level of the epigenome. In certain cases, the epigenome modifying polypeptide comprises a DNA methyltransferase domain. The term “DNA methyltransferase” as provided herein refers to an enzyme that catalyzes the transfer of a methyl group to DNA. Non-limiting examples of DNA methyltransferases include Dnmt1, Dnmt3A, Dnmt3B, and Dnmt3L. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors or CTCF. As described herein, fusion proteins may include one or more (e.g., two) DNA methyltransferases. When a DNA methyltransferase is included as part of a fusion protein, the DNA methyltransferase may be referred to as a “DNA methyltransferase domain.” In aspects, a DNA methyltransferase domain includes one or more DNA methyltransferases. In aspects, a DNA methyltransferase domain includes two DNA methyltransferases.

TABLE 1
LNP formulations for brain delivery: F5, L-4, L-5, L-6, L-10, L-14, L-18, L-24,
L-25, L-36, L-40, L-42, L-45, L-46, and L-47.

| | | |
|---|---|---|
| F5 Dlin DOPE Cholesterol DMG PEG Cerebrosides | % 30.787 20.838 32.144 1.231 14.999 | 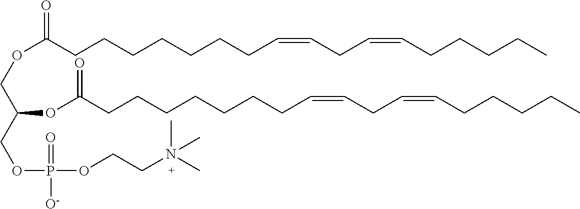 |
| L-4 Lipid 5 DOPE Cholesterol DMG PEG | % 34.491 24.692 39.596 1.220 | 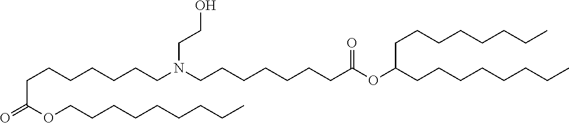 [SMILES: CCCCCCCCCOC(=O)CCCCCCCN(CCO)CCCCCCCC(=O)OC(CCCCCCCC)CCCCCCCC] |
| L-5 L319 DOPE Cholesterol DMG PEG | % 35.547 24.294 38.958 1.201 | 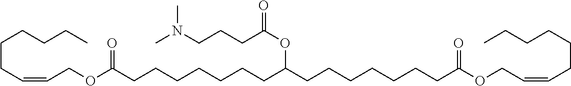 [SMILES: CCCCCC/C=C\COC(=O)CCCCCCCC(CCCCCCCC(=O)OC/C=C\CCCCCC)OC(=O)CCCN(C)C] |
| L-6 Lipid A6 DOPE Cholesterol DMG PEG | % 34.751 24.594 39.439 1.215 | 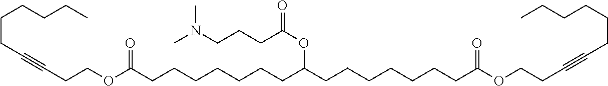 [SMILES: CCCCCCC#CCCOC(=O)CCCCCCCC(CCCCCCCC(=O)OCCC#CCCCCCC)OC(=O)CCCN(C)C] |
| L-10 cKK-E12 DOPE Cholesterol DMG PEG | % 34.466 24.702 39.611 1.221 | 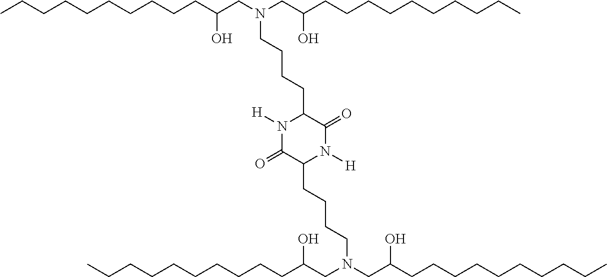 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC] |
| L-14 Lipid C24 DOPE Cholesterol DMG PEG | % 34.780 24.583 39.422 1.215 | 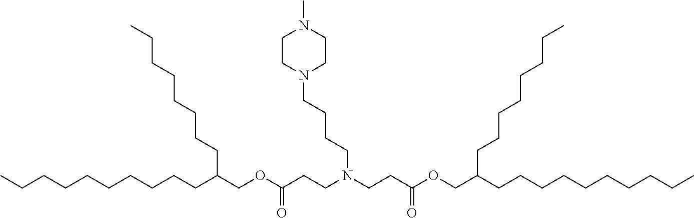 [SMILES: CCCCCCCCCCC(CCCCCCCC)COC(=O)CCN(CCCCN1CCN(C)CC1)CCC(=O)OCC(CCCCCCCC)CCCCCCCCCC] |
| L-18 ALC-0315 DOPE Cholesterol DMG PEG | % 34.512 24.684 39.583 1.220 | 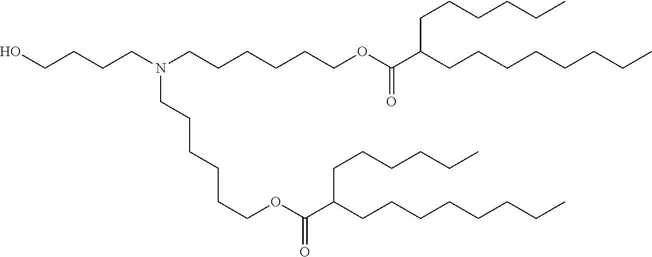 [SMILES: CCCCCCCCC(CCCCCC)C(=O)OCCCCCCN(CCCCO)CCCCCCOC(=O)C(CCCCCC)CCCCCCCC] |
| L-24 OF-02 DOPE Cholesterol DMG PEG | % 34.791 24.579 39.415 1.215 | 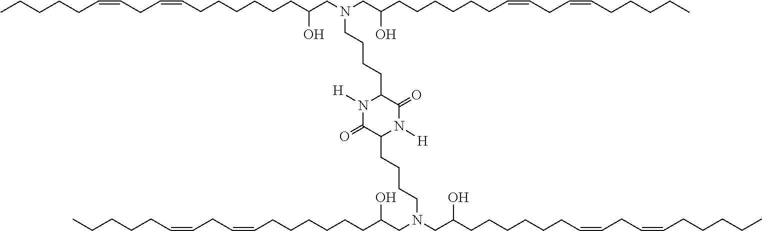 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| L-25 Lipid A9 DOPE Cholesterol DMG PEG | % 34.491 24.692 39.596 1.220 | 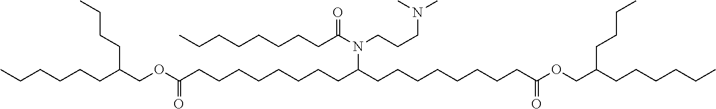 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| L-36 Lipid CL1 DOPE Cholesterol DMG PEG | % 34.411 24.722 39.645 1.222 | 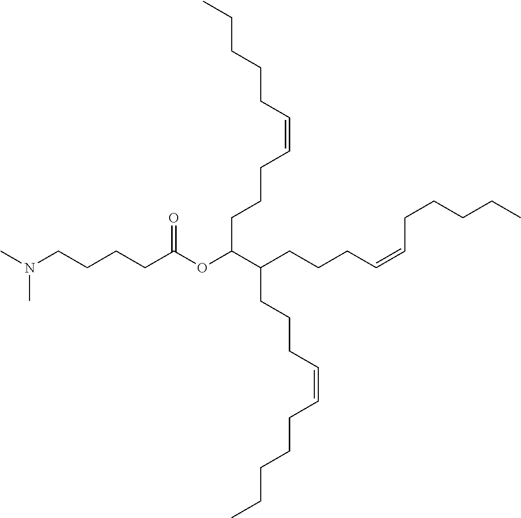 [SMILES: CCCCC/C=C\CCCC(CCC/C=C\CCCCC)C(CCC/C=C\CCCCC)OC(=O)CCCCN(C)C] |
| L-40 YK-009 DOPE Cholesterol DMG PEG | % 34.589 24.655 39.537 1.218 | 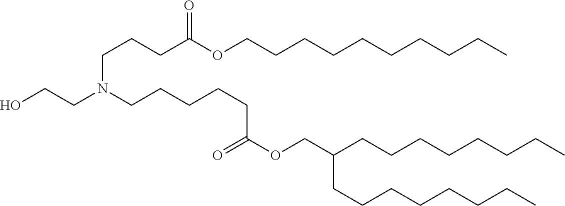 [SMILES: CCCCCCCCCCOC(=O)CCCN(CCO)CCCCCC(=O)OCC(CCCCCCCC)CCCCCCCC] |
| L-42 ALC-0315 analogous-1 DOPE Cholesterol DMG PEG | % 34.624 24.642 39.516 1.218 | 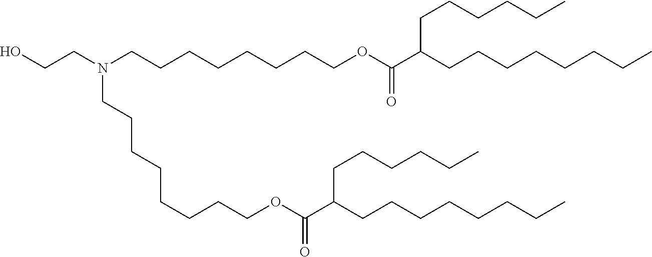 [SMILES: CCCCCCCCC(CCCCCC)C(=O)OCCCCCCCCN(CCO)CCCCCCCCOC(=O)C(CCCCCC)CCCCCCCC] |
| L-45 AA-T3A- C12 DOPE Cholesterol DMG PEG | % 34.611 24.647 39.524 1.218 | 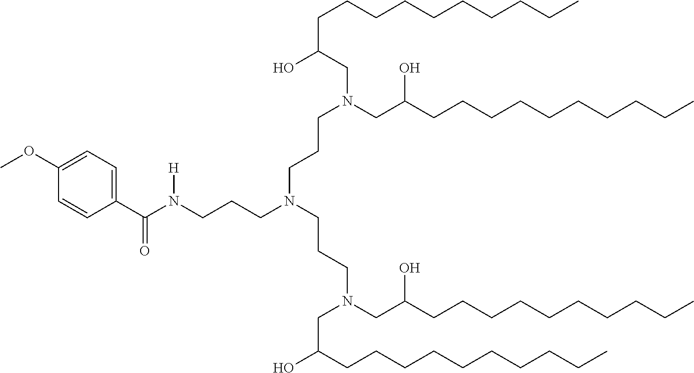 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC] |
| L-46 CL4F8-6 DOPE Cholesterol DMG PEG | % 34.476 24.698 39.605 1.221 | 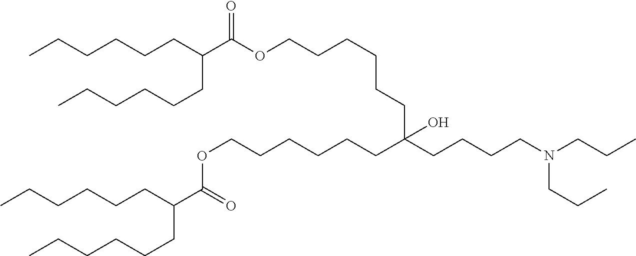 [SMILES: CCCCCCC(CCCCCC)C(=O)OCCCCCCC(O)(CCCCCCOC(=O)C(CCCCCC)CCCCCC)CCCCN(CCC)CCC] |
| L-47 Lipid III-45 DOPE Cholesterol DMG PEG | % 34.608 24.648 39.525 1.218 | 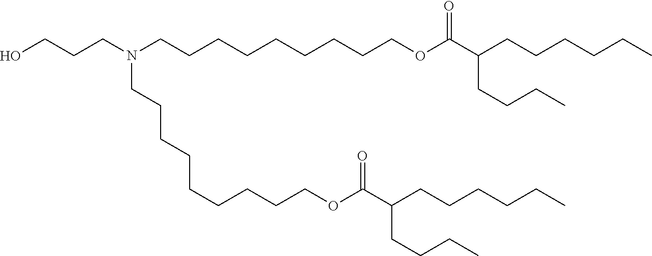 [SMILES: CCCCCCC(CCCC)C(=O)OCCCCCCCCCN(CCCO)CCCCCCCCCOC(=O)C(CCCC)CCCCCC] |

1. A composition comprising a lipid nanoparticle (LNP) configured for delivering a nucleic acid to the brain, the LNP comprising a cerebroside in a bilayer forming ionizable lipid comprising an acid degradable polyethylene glycol (PEG), and gene editing components and a gRNA targeting an endogenous locus.

2. The composition of claim 1, comprising an azide-benzaldehyde acetal cholesterol.

3. The composition of claim 1, wherein the ionizable lipid is selected from: Dlin, Lipid 9, L-319, L-A6, cKK-E12, Lipid C24, ALC-0315, OF-02, Lipid A9, Lipid CL1, YK-009, ALC-0315 analogous-1, AA-T3A-C12, CL4F8-6, Lipid III-45.

4. The composition of claim 1, further comprising a helper lipid, such as DOPE, cholesterol, and a DMG PEG (dimyristoyl glycerol polyethyleneglycol).

5. The composition of claim 1, further comprising a cerebroside, including monoglycosyl and oligoglycosylceramides having a mono or polysaccharide bonded glycosidically to the terminal OH group of ceramide, with sphingosine as the main long-chain base present in the ceramide.

6. The composition of claim 1, comprising a lipid formulation selected from: F5, L-4, L-5, L-6, L-10, L-14, L-18, L-24, L-25, L-36, L-40, L-42, L-45, L-46, and L-47, herein.

7. The composition of claim 1, comprising a lipid formulation of F5-PEG, herein.

8. The composition of claim 1, wherein the gene editing components are selected from CRISPR/Cas systems, Zinc Finger Nuclease (ZFN), Transcription activator-like effector nucleases (TALENs), and Meganucleases, Mega-TALS.

9. The composition of claim 1, wherein the gene editing components are a CRISPR/Cas system.

10. The composition of claim 1, wherein the endogenous locus is microtubule-associated protein tau (MAPT).

11. The composition of claim 1, wherein:
the gene editing components are CRISPR/Cas9;
the endogenous locus is microtubule-associated protein tau (MAPT); and
the LNP comprises a lipid formulation of F5-PEG, herein.

12. A method of in vivo gene editing comprising delivering in vivo by intracranial injection. to a neurons of a central nervous system a composition of claim 1, to prevent or treat brain diseases like Alzheimer's disease or Frontotemporal Dementia.


SEQUENCE LISTING (8 sequences)

SEQ ID NO: 1 - DNA, 40 bp, synthetic construct
  source 1..40: mol_type = other DNA
tgacgagaagaaagccaaggtaagctactgacccgccggt

SEQ ID NO: 2 - DNA, 40 bp, synthetic construct
  source 1..40: mol_type = other DNA
cacaccattagttgtcagggaaatgcaaatcaaaccacag

SEQ ID NO: 3 - DNA, 35 bp, synthetic construct
  source 1..35: mol_type = other DNA
tgacgagaagaaagccaagctactgacccgccggt

SEQ ID NO: 4 - DNA, 39 bp, synthetic construct
  source 1..39: mol_type = other DNA
tgacgagaagaaagccaaggaagctactgacccgccggt

SEQ ID NO: 5 - DNA, 40 bp, synthetic construct
  source 1..40: mol_type = other DNA
tgacgagaagaaagccaaggttaagctactgacccgccgg

SEQ ID NO: 6 - DNA, 38 bp, synthetic construct
  source 1..38: mol_type = other DNA
tgacgagaagaaagccaataagctactgacccgccggt

SEQ ID NO: 7 - DNA, 30 bp, synthetic construct
  source 1..30: mol_type = other DNA
tgacgagaagaaagctactgacccgccggt

SEQ ID NO: 8 - DNA, 35 bp, synthetic construct
  source 1..35: mol_type = other DNA
tgacgagaagaaagccaaggtactgacccgccggt